(19) United States
(12) Patent Application Publication
(10) Pub. No.: US 2026/0215888 A1
(43) Pub. Date: Jul. 30, 2026

(54) DENTAL IMPLANT SYSTEM

(71) Applicant: Randall F. Lee, Southlake, TX (US)

(72) Inventors: Randall F. Lee, Southlake, TX (US); Ian A. Lee, Carrollton, TX (US); Matthew Shomper, Xenia, OH (US); Lisa Ferrara, Hendersonville, NC (US)

(73) Assignee: Randall F. Lee, Southlake, TX (US)

(21) Appl. No.: 19/573,940

(22) Filed: Mar. 20, 2026

Related U.S. Application Data
(63) Continuation of application No. PCT/US2025/028519, filed on May 8, 2025.
(60) Provisional application No. 63/644,222, filed on May 8, 2024.
(60) Provisional application No. 63/677,230, filed on Jul. 30, 2024.

(51) Int. Cl. A61C 8/00

(52) U.S. Cl. CPC A61C 8/0025; A61C 8/0039; A61C 8/0096; A61C 2008/0046; A61C 2008/0084

(57) ABSTRACT

Embodiments of an implant fixture for a dental implant device are disclosed, including a shaft having a first rotational axis; a propeller-like region comprising helical threads positioned about a longitudinal portion of the shaft forming a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; and an irregular lattice structure formed within the plurality of helical root valleys.

### CROSS-REFERENCE TO RELATED APPLICATIONS

This application is a continuation of International Application No. PCT/US2025/028519, filed May 8, 2025, entitled “DENTAL IMPLANT SYSTEM,” which claims the benefit of the filing date of U.S. provisional patent application Ser. No. 63/644,222, filed on May 8, 2024, entitled “SYSTEM AND METHOD FOR A MEDICAL IMPLANT WITH AN INTEGRATED PROPULSOR,” and U.S. provisional patent application Ser. No. 63/677,230, filed on Jul. 30, 2024, entitled “SYSTEM AND METHOD FOR A MEDICAL IMPLANT WITH AN INTEGRATED PROPULSOR, the disclosures of which are incorporated herein by reference for all purposes.

### TECHNICAL FIELD

The invention relates in general to dental implant systems and, in particular, to dental systems with self-compressing propulsor-shaped implants.

### BACKGROUND INFORMATION

Dental implants have revolutionized restorative dentistry, providing a predictable and effective treatment option for replacing missing teeth. The long-term success of dental implants relies heavily on osseointegration, the direct structural and functional connection between living bone and the implant surface. While modern dental implants boast high success rates, challenges remain, particularly in achieving and maintaining optimal osseointegration in all patients and clinical scenarios.

One of the primary limitations of current implant technologies lies in the variability of osseointegration rates and quality, especially in compromised bone conditions. Factors such as poor bone density (e.g., in patients with osteoporosis or after tooth extraction), previous bone grafting procedures, and systemic conditions (e.g., diabetes, smoking) can significantly impair the osseointegration process, leading to delayed healing, reduced implant stability, and increased risk of early implant failure.

Furthermore, achieving predictable osseointegration in immediate loading protocols remains a challenge. While immediate loading offers advantages in terms of reduced treatment time and improved patient satisfaction, it places higher demands on initial implant stability and the rate of bone formation around the implant. The risk of micromovement and subsequent fibrous encapsulation is higher with immediate loading, potentially compromising long-term osseointegration.

Another area of concern is the long-term maintenance of osseointegration in the face of peri-implantitis. This inflammatory condition, characterized by bone loss around the implant, can lead to implant loosening and eventual failure. While various treatment strategies exist for peri-implantitis, prevention is paramount, and enhanced osseointegration may contribute to improved resistance to peri-implant disease progression.

Current implant surface modifications, such as acid etching and sandblasting, have significantly improved osseointegration compared to earlier smooth-surface implants. However, there is still room for improvement in terms of accelerating the osseointegration process, enhancing bone-to-implant contact, and promoting a more robust and resilient bone-implant interface, particularly in challenging clinical situations.

What is needed, therefore, is a device or system that can enhance osseointegration, improve predictability in compromised bone, and promote long-term implant stability.

### SUMMARY

In response to these and other problems, in one embodiment, there is a dental implant system comprising a propulsor-shaped implant body or fixture, an abutment, and an abutment connector.

In certain embodiments, the implant fixture comprises a shaft having a first rotational axis; helical threads positioned about a longitudinal portion of the shaft forming a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; an irregular lattice structure formed within the plurality of helical root valleys; and a means for attaching the abutment to the implant fixture.

In embodiments described above, a pawl in the plurality of pawls has an angled surface facing a forward rotation direction and a concave curved surface facing away from the forward rotation direction.

In the embodiments described above, a pawl in the pluralities of pawls has a sharp edge projecting away from the direction of forward rotation and towards the direction of backward rotation.

In embodiments described above, a pawl in the plurality of pawls has an angled surface facing a forward rotation direction and a concave curved surface facing away from the forward rotation direction.

In embodiments described above, a pawl in the plurality of pawls has an angled surface facing a forward rotation direction and a straight surface facing away from the forward rotation direction.

In embodiments described above, a pawl in the plurality of pawls has an angled surface facing a forward rotation direction and an angled surface facing away from the forward rotation direction.

In embodiments described above, the threads include a circumferential rim projecting from the proximal surface of the threads.

In embodiments described above, the proximal surface of the threads is curved in a concave manner.

In other embodiments, there may be at least one longitudinal channel defined within the propulsor having an intake aperture defined in a proximal portion of the propulsor and at least one output aperture defined along the shaft positioned distally from the intake aperture.

In other embodiments, there may be at least one longitudinal channel defined within the propulsor, having an intake aperture defined in a proximal portion of the propulsor and a plurality of tubules branching from at least one longitudinal channel and ending in a plurality of output apertures defined along the shaft and positioned distal to the intake aperture.

In some embodiments discussed above, the cross-sectional area of the longitudinal channel varies along its longitudinal length.

In some embodiments discussed above, the cross-section areas of the tubules are variable in size to allow for a uniform deposition of material through the plurality of output apertures.

In some embodiments discussed above, an intake aperture is defined within a central bore located defined within the proximal end.

In the embodiments described above, the propulsor comprises a distal portion and a proximal portion, wherein the distal portion includes a forward distal thread-form shape to assist in drilling through bony tissue during implant placement.

In yet other embodiments, there may be an implant fixture for a dental implant device, comprising: a propulsor comprising a shaft having a first rotational axis; helical threads positioned about a longitudinal portion of the shaft forming a plurality of helical root valleys between crests of the helical threads, and the helical threads having distal surfaces and proximal surfaces; a plurality of pawls projecting from the distal surfaces to create a turbine-like thread-form shape; wherein the plurality of pawls are angled with respect to the distal surfaces to allow for forward rotation of the threads and to resist backward rotation of the threads; and an irregular lattice structure formed within the plurality of helical root valleys.

In yet other embodiments, there may be a method of placing an implant fixture in bony material, the method comprising: applying a torque to a proximal end of a propulsor to rotate a propulsor of the surgical implant about the propulsor's longitudinal axis in a first rotational direction within a first bony structure to propel the implant in a first longitudinal direction; rotating a first plurality of rear-facing pawls in the first rotational direction to compress the bony material by the pawls while propelling the propulsor forward; compressing bony material between a cupped proximal side of a first thread and the distal side of a second thread; and injecting a first internal labyrinth defined in the first propulsor with bone growth material such that the bone-grown material flows from an opening defined in a proximal portion of the propulsor into a plurality of channels and out of a plurality of openings defined along the shaft.

These and other features and advantages will be more clearly understood from the following detailed description taken in conjunction with the accompanying drawings. It is important to note that the drawings are not intended to represent the only aspect of the invention.

### BRIEF DESCRIPTION OF THE DRAWINGS

FIG. 1A is a perspective view illustrating one embodiment of an implant system that incorporates one or more aspects of the present invention.

FIG. 1B is a sectional view of the implant system illustrated in FIG. 1A.

FIG. 2A is a perspective view illustrating one embodiment of an implant system kit, illustrating the range of sizes of implants.

FIG. 2B is a sectional perspective view of the implant system kit illustrated in FIG. 2A.

FIG. 3A is a perspective view illustrating one embodiment of an implant fixture that incorporates one or more aspects of the present invention.

FIG. 3B is a distal end view of the implant fixture illustrated in FIG. 3A.

FIG. 3C is a perspective side view of the implant fixture illustrated in FIG. 3A.

FIG. 3D is a perspective sectional view of the implant fixture illustrated in FIG. 3A cut along a center axis.

FIG. 3E is a detailed perspective view of a thread form embodiment, which may be employed by the implant fixture illustrated in FIG. 3A.

FIG. 3F is a perspective sectional view of the implant fixture illustrated in FIG. 3A cut through a plurality of thread forms.

FIG. 3G is a detailed perspective sectional view of the implant fixture illustrated in FIG. 3A cut through a single thread form.

FIG. 3H is a detailed perspective view of an alternative implant feature showing edge details of a thread.

FIG. 3I is a detailed perspective view of the proximal faces of a plurality of thread forms, which may be used by the implant fixture illustrated in FIG. 3A.

FIG. 3J is a perspective sectional view of the implant fixture illustrated in FIG. 3A, cut along a center axis, where the interior material is illustrated as transparent to show one embodiment of internal cannulas and passageways.

FIG. 3K is a side view of the implant fixture illustrated in FIG. 3A, where the material forming the fixture is illustrated as semi-transparent to show one embodiment of the internal cannulas and passageways.

### DETAILED DESCRIPTION

For the purposes of promoting an understanding of the principles of the present inventions, reference will now be made to the embodiments or examples illustrated in the drawings, and specific language will be used to describe the same. It will nevertheless be understood that no limitation of the scope of the invention is thereby intended. Any alterations and further modifications in the described embodiments and any further applications of the principles of the inventions as described herein are contemplated as would normally occur to one skilled in the art to which the invention relates.

When directions, such as upper, lower, top, bottom, clockwise, or counter-clockwise, are discussed in this disclosure, such directions are meant to only supply reference directions for the illustrated figures and for orientation of components with respect to each other or to illustrate the figures. The directions should not be read to imply actual directions used in any resulting invention or actual use. Under no circumstances should such directions be read to limit or impart any meaning into the claims.

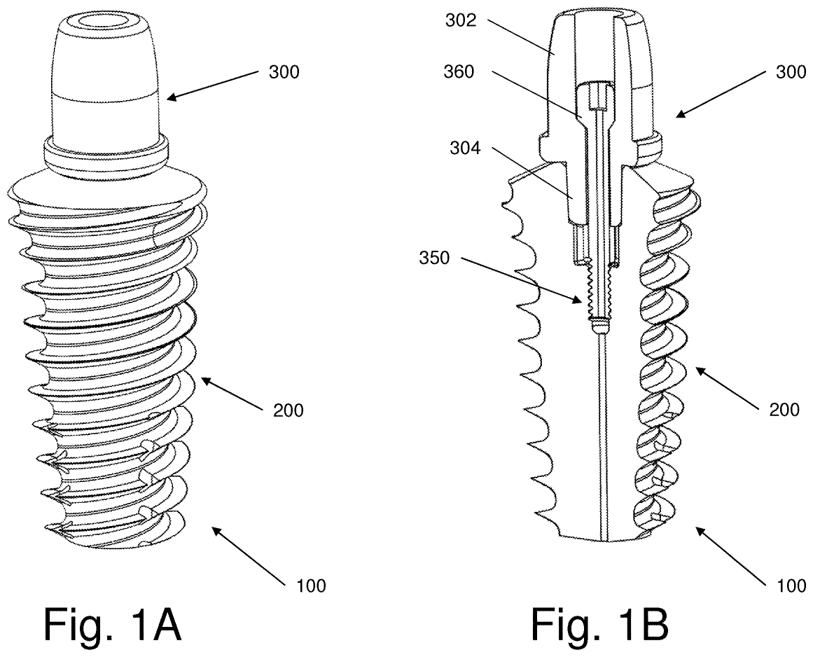

Turning now to FIGS. 1A and 1B, there is presented one embodiment of an example dental implant system 100 illustrating an implant fixture 200, an abutment 300, and an abutment attachment mechanism 350. FIG. 1B illustrates a sectional view of the dental implant system 100, showing a sectional view of the implant fixture 200, an abutment 300, and an abutment attachment mechanism 350.

In certain embodiments, the implant fixture 200 is designed to be surgically placed into the jawbone. The implant fixture 200 may be considered the “root” of the implant system 100. As will be explained in more detail below, after placement, the implant fixture 200 provides a surface for the surrounding bone to grow onto and integrate with, creating a stable foundation and retention for the abutment 300. The implant fixture 200 also transfers occlusal forces from the final prosthesis (i.e., crown) and the abutment 300 to the surrounding bone.

In certain embodiments, the abutment 300 comprises an abutment body 302 and a connection interface 304. The abutment body 302 is the main portion that connects to and supports a prosthesis (e.g., a crown, bridge, or denture). The abutment body 302 varies in shape and size depending on the clinical needs. A connection interface 304 is part of the abutment 300 designed to fit into the implant fixture 200. In certain embodiments, an internal connection, such as an internal hex or Morse taper, may be used to join the connection interface 304 to the implant fixture 200.

In certain embodiments, the abutment 300 serves as a connector that attaches the implant fixture 200 to the prosthesis or restoration component (e.g., a crown, bridge, or denture). The abutment 300 may also act as an interface between the implant fixture 200 and the final prosthesis (not shown). The margin of the abutment 300 (where it meets the restoration) can be positioned at different levels relative to the patient's gum line, such as equigingival or subgingival, based on functional and aesthetic needs. In certain embodiments, the abutment 300 not only connects the implant fixture 200 to the prosthesis (not shown) but also provides support and retention for the prosthesis. In certain embodiments, the abutment 300 may also assist in creating a natural-looking emergence profile, enhancing the prosthesis's appearance to resemble a natural tooth. In yet other embodiments, the abutment 300 can be angled to correct for non-ideal implant fixture placements. Additionally, the abutment 300 may also aid the management of surrounding soft tissues, helping to shape and support the gums.

In certain embodiments, the abutment 300 may be crafted from various biocompatible materials, such as titanium, zirconia, gold alloy, or even ceramics. The illustrative embodiment of the abutment 300 is an example. Other abutment embodiments may be provided in diverse shapes and sizes to suit different clinical scenarios and are within the scope of this invention. In certain embodiments, the abutment 300 may be pre-fabricated (i.e., a stock abutment) or custom-milled to meet the specific requirements of the patient.

An internal connection mechanism 350 may be used to connect the abutment 300 to the implant fixture 200 and is designed to provide a secure and precise fit between the abutment and implant fixture. In certain embodiments, an internal hex connection (not shown) may be used as the connection mechanism, where a hexagonal recess is incorporated into the top of the implant fixture, allowing the abutment 300 to be inserted and twisted into place. This design may provide rotational stability, a reduction in micromovement, and assist in maintaining the position of the abutment during function and use.

In other embodiments, a Morse taper connection may be used. A Morse taper connection involves a conical, friction-fit interface. In this embodiment, the abutment 300 fits snugly into a matching tapered socket inside the implant fixture, which may create a tight seal that minimizes microleakage and enhances stability.

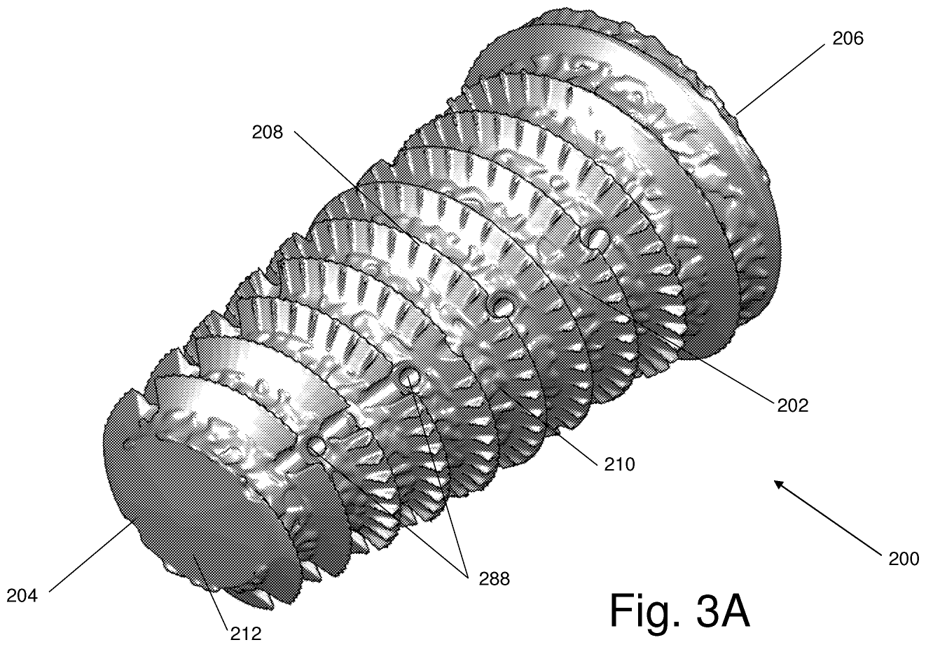

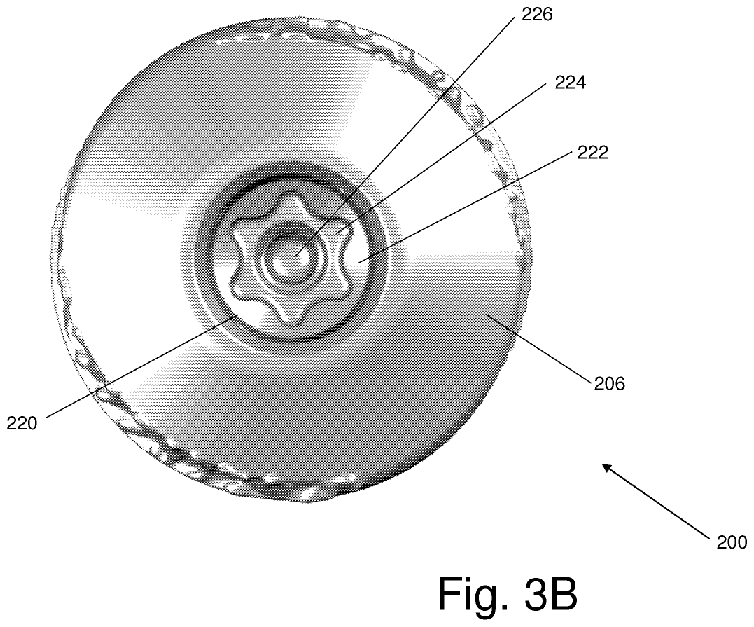

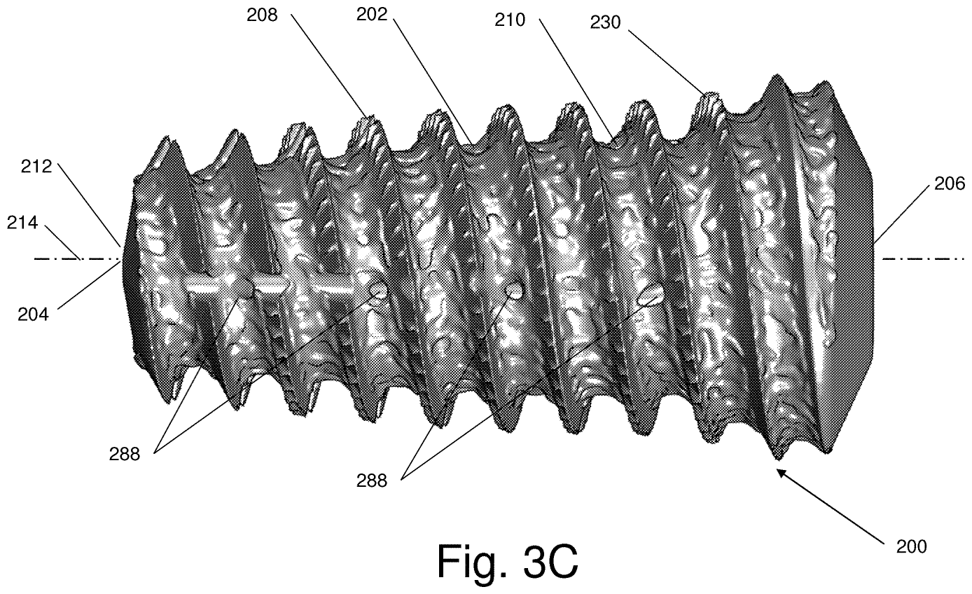

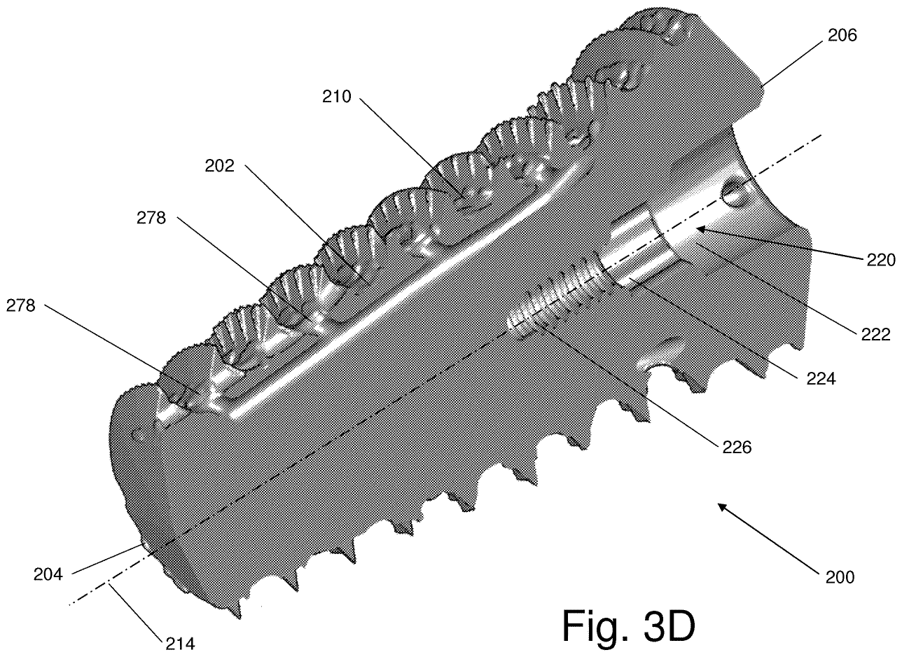

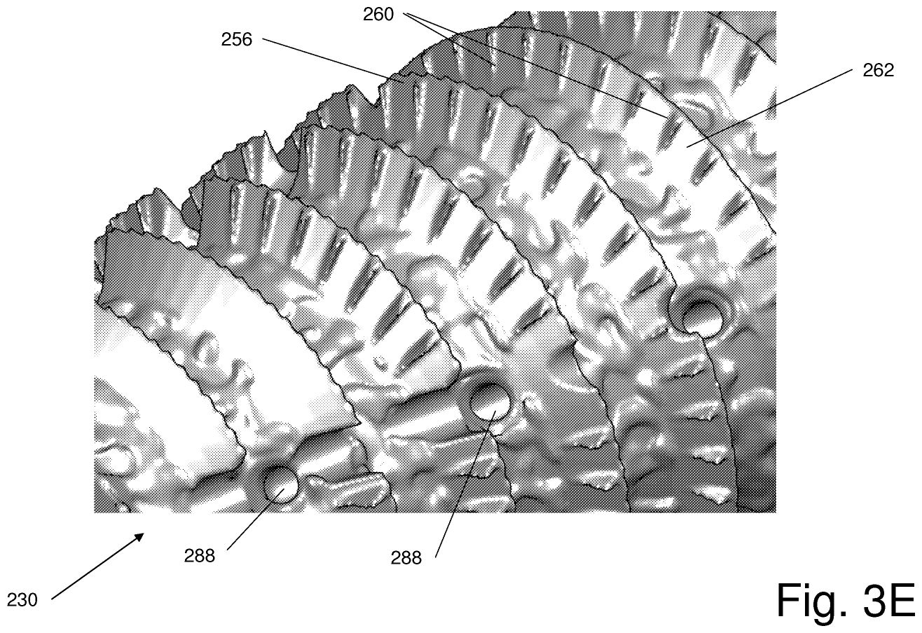

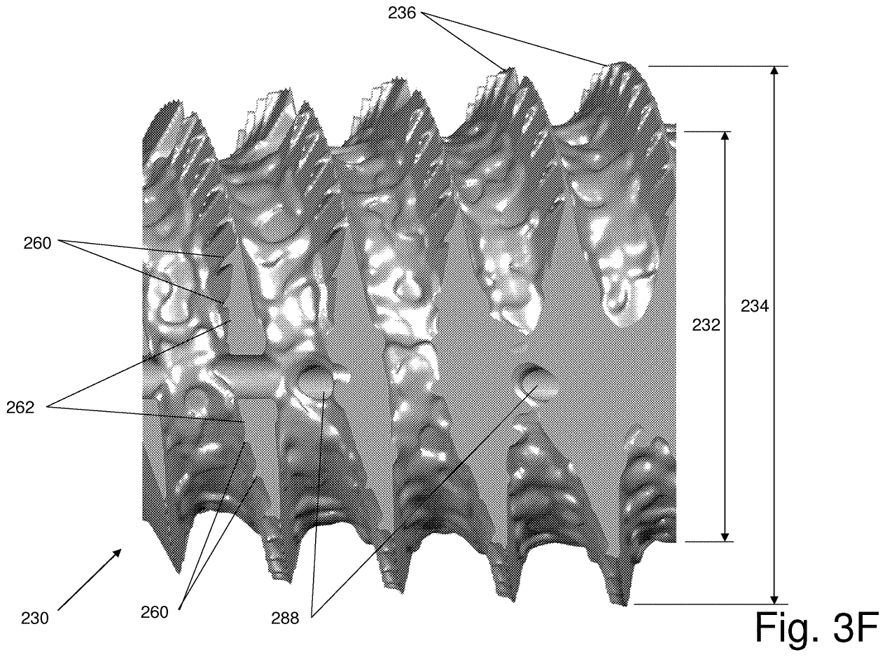

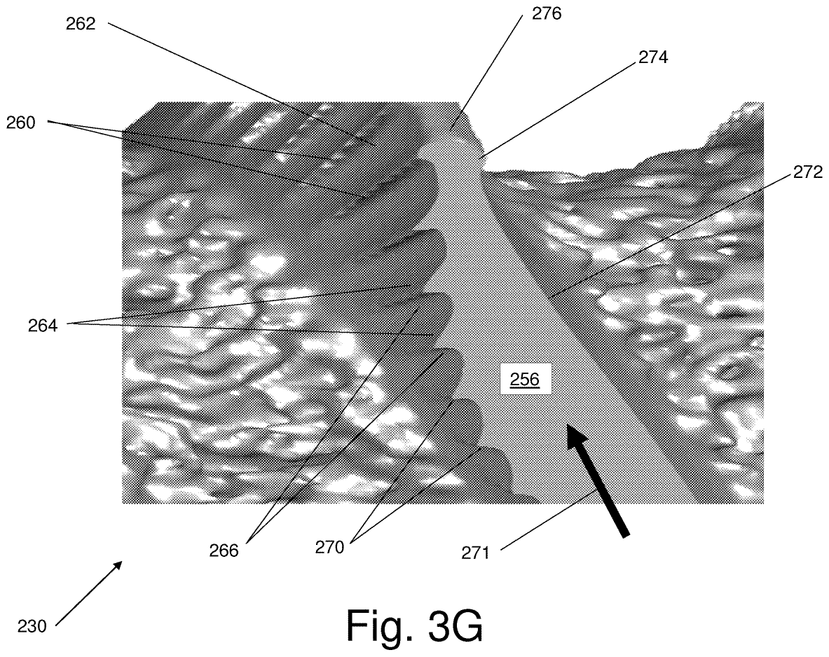

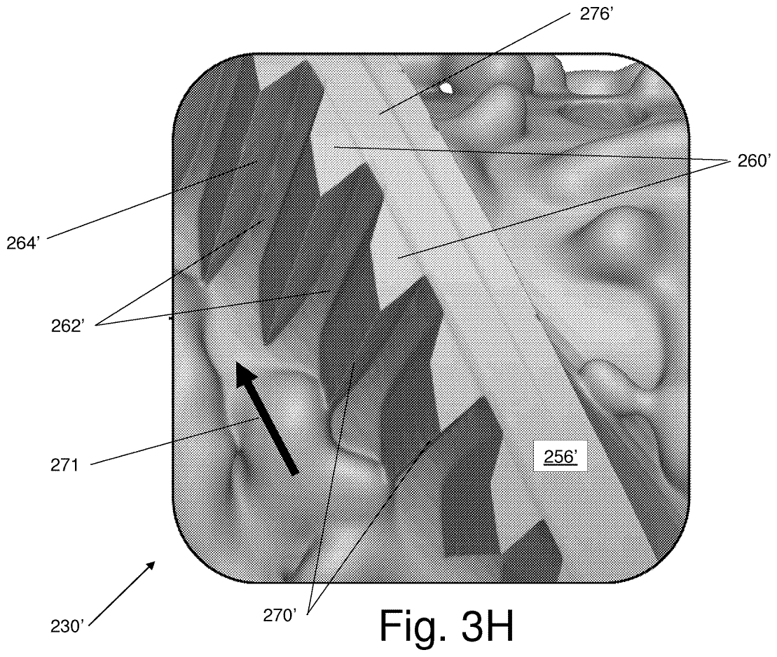

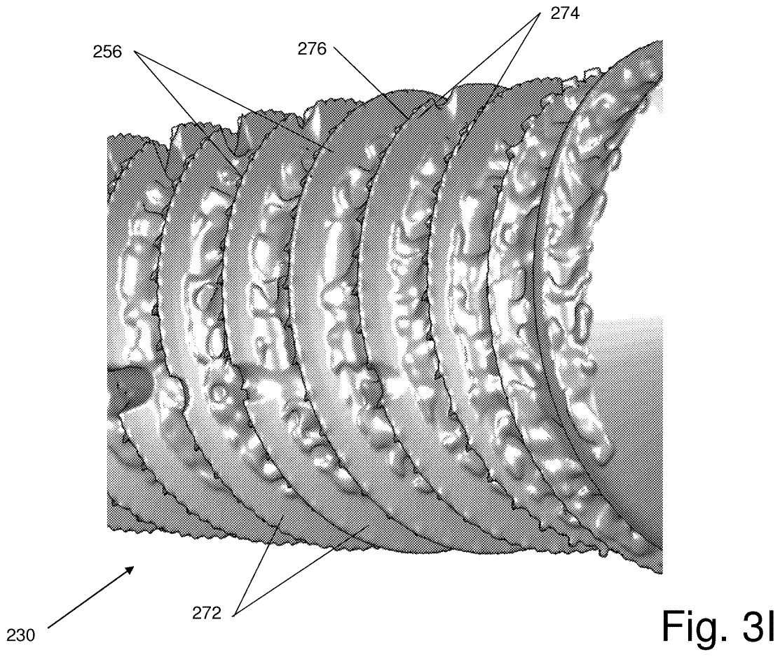

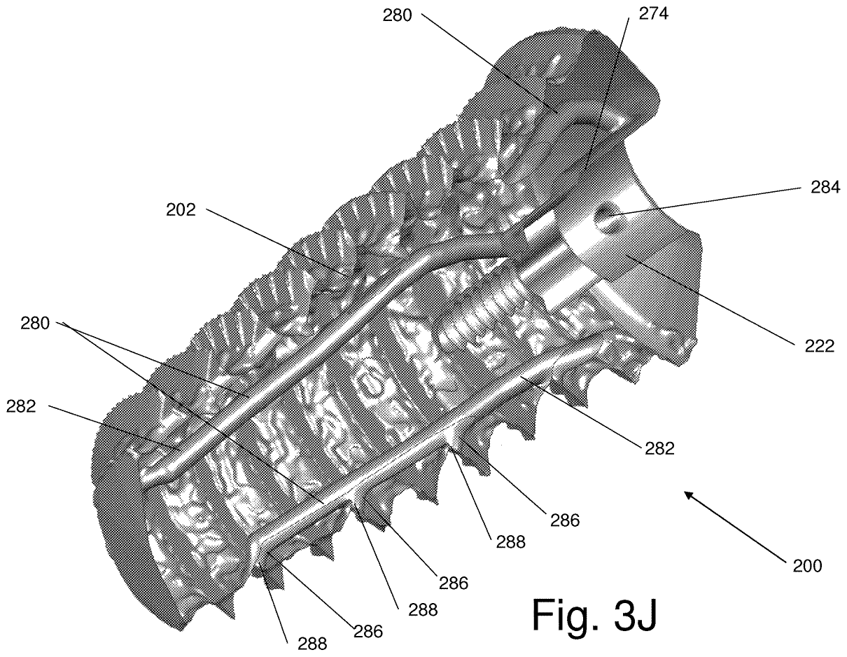

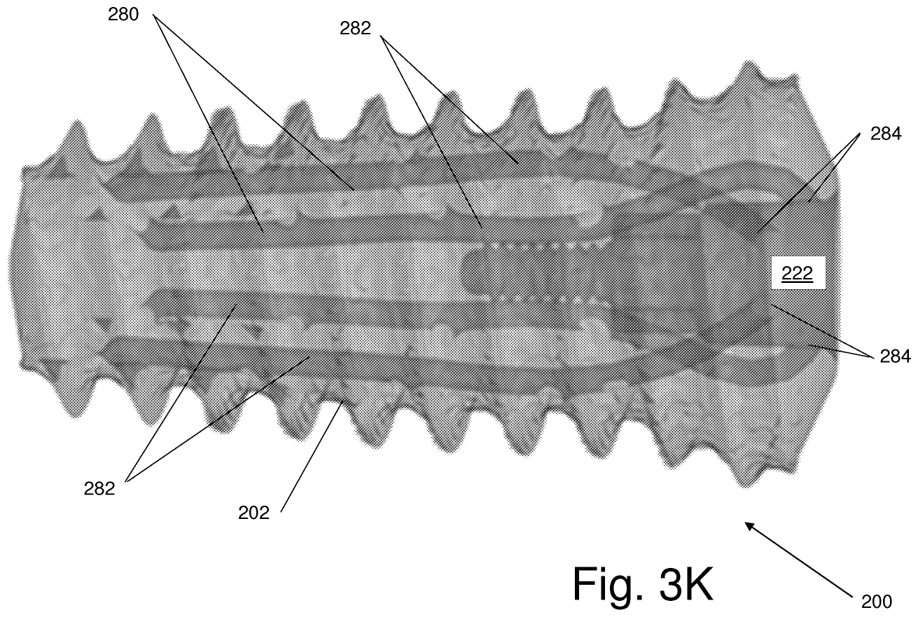

In certain embodiments, the abutment 300 may be secured to the implant fixture 200 using a connector known in the art, such as an abutment screw 360, which may be inserted through the top of the abutment 300 into the implant fixture's internal threaded connection (i.e., a threaded portion 226 of FIG. 3D below). When tightened, the abutment screw 360 locks the abutment 300 firmly in place, ensuring it does not loosen under functional forces. Some embodiments may also incorporate additional features like indexing or anti-rotational elements to prevent screw loosening and to facilitate proper orientation during placement.

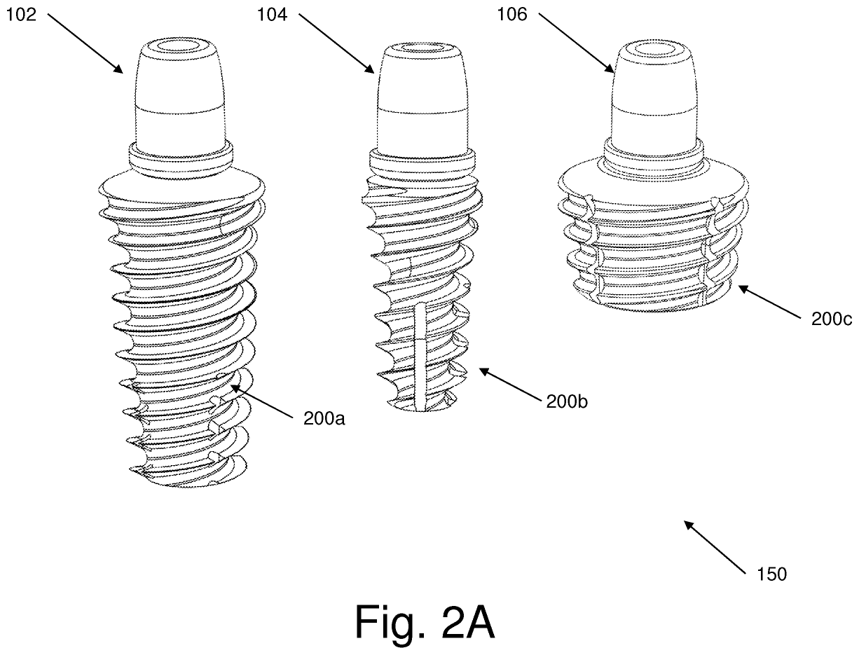

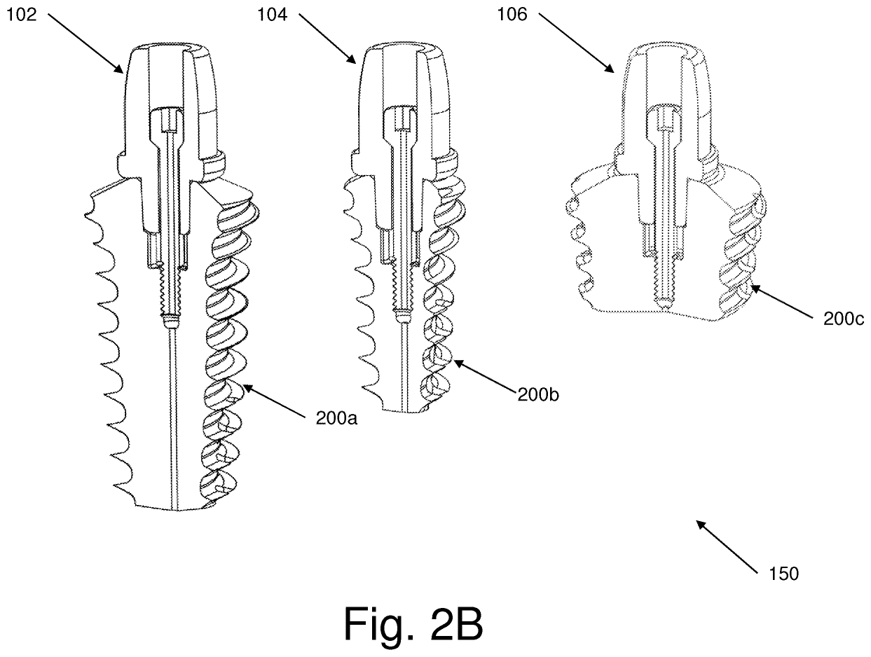

FIG. 2A illustrates a dental implant kit 150 comprising a variety of different-sized implant systems 102, 104, and 106. FIG. 2B illustrates the kit of FIG. 2A with implant systems shown in a sectional view to show the relative sizes of the internal components. In the kit 150 illustrated in FIGS. 2A and 2B, the implant fixtures 200a, 200b, and 200c are illustrated in three different sizes. As an example, in the implant system 102, the implant fixture 200a may have a length of 11 mm and an outside diameter of 4.5 mm. In the implant system 104, the implant fixture 200b may have a length of 8 mm and an outside diameter of 3 mm. In the implant system 106, the illustrated implant fixture 200c may have a length of 6 mm and an outside diameter of 5 mm. Of course, the sizes discussed in the implant systems of the kit 150 are meant to be examples only and not meant to restrict or impose limitations on the scope of the claims. Furthermore, the number of implant systems and implant fixtures illustrated are just meant to illustrate the concept. Actual kits may have a greater or lesser number of implant systems or implant fixtures.

In addition to the illustrated implant fixtures 200a-200c, embodiments of the implant fixture 200 may be produced in a variety of other shapes and sizes to accommodate the diverse anatomical and clinical conditions encountered in dental implantology. Different patients present with varying bone densities, volumes, and geometries, as well as unique functional and aesthetic requirements, making a one-size-fits-all approach impractical. By offering a range of implant designs—such as varying diameters, lengths, and body contours—clinicians can select the most appropriate fixture to achieve optimal primary stability, distribute occlusal forces effectively, and preserve surrounding bone structures. Additionally, certain implant shapes are better suited for specific locations within the mouth or for particular types of restorations, such as short or wide implants for atrophic ridges or angled fixtures for complex cases.

### The Features of the Implant Fixture or Body:

FIG. 3A is a perspective view of the implant fixture 200 as viewed from its distal end 204. FIG. 3B is an end view of the implant fixture 200 as viewed from a proximal end 206, and FIG. 3C is a side view of the implant fixture 200.

In certain embodiments, the implant fixture 200 comprises a center longitudinal tapered shaft 202, as illustrated in FIGS. 3A and 3C. The taper starts from the proximal end 206, having a first diameter, and narrows to the distal end 204, which has a second or smaller diameter. In certain embodiments, the taper allows the shaft 202 to create a wedging action as it is driven into a bony structure (not shown), offering a secure and tight fit after placement. This gradual change in diameter may also help to ensure greater holding power and better centering when compared to embodiments with a constant diameter.

As will be explained below in greater detail, in certain embodiments, one or more threads or thread flights 208 circumferentially encircle the shaft 202 in a helical manner, creating a series of helical root valleys and crests as illustrated in FIG. 3C. In certain embodiments, as best illustrated in FIGS. 3A and 3C, there may be a porous lattice or fenestrated structure 210 may be etched, milled, or three-dimensionally (3D) printed around the shaft 202 in the valleys or minor diameter (between the threads) of the implant fixture 200 to encourage bone growth. In embodiments, which include the internal labyrinth discussed below, the cannulas and channels of the labyrinth may end in apertures 288 defined in this lattice 210 (or slightly beyond the lattice) to further encourage bone growth in and around the shaft.

In certain embodiments, the distal end 204 of shaft 202 may have a conical or cutting surface 212 defined therein to penetrate and allow movement through bony tissue (not shown). In certain embodiments, a center bore 220 may be defined at the proximal end 206 of the center shaft 202 as illustrated in FIGS. 3B and 3D. In certain embodiments, the central bore 220 runs towards the distal end 204 along a longitudinal axis 214.

FIG. 3D is a perspective section view of one embodiment of the implant fixture 200, showing a section cut approximately along the longitudinal or center axis 214 of the center shaft 202 and through the center bore 220. In certain embodiments, the center bore 220 comprises three sections: a proximal circular receiving aperture 222, a torque engagement section or feature 224, and a threaded portion 226. The torque engagement section 224 is defined within the center bore 220 for engaging with a torque-inducing or driving device (not shown). For instance, in some embodiments, the torque engagement feature 224 may be a torx-shaped socket for engaging a similarly shaped torx driver of an insertion or driving instrument (not shown). In other embodiments, the engagement feature 224 may be hexagonal, octagonal, or trilobe, depending on the specific application and shape of the insertion instrument.

As described above, the threaded portion 226 is sized to receive the abutment screw 360 (not shown).

As illustrated in FIGS. 3A and 3C, in certain embodiments, there may be helical threads 208 or a helical flight of threads having unique characteristics and features, which will be explained below. The characteristics of the helical threads are designed to have an increased propulsion force to propel the implant fixture 200 in a forward direction while creating compression in the surrounding bony tissue during and after placement. Thus, this helical flight creates greater propulsion and compression. For purposes of this disclosure, this “region of increased propulsion and compression” will be referred to as a propeller region or “propeller” 230.

FIG. 3E is a detailed perspective view illustrating an example of aggressive helical thread form geometry, which acts as the propeller 230. FIG. 3F is a partial section view of the helical thread form of a thread in the propeller region 230, where the section is cut through the threads to illustrate the unique shape of the thread forms in the propeller region.

In certain embodiments, as illustrated in FIG. 3F, there may be an aggressive “propeller” design characterized by relatively high crests 236 of the implant thread form showing a large radial difference between the minor diameter 232 and the major diameter 234, which may be relatively large compared to the prior art. This difference helps to maximize the propulsive force as the propeller 230 propels the implant fixture 200 into the bony material and provides the compression of the bony material as the implant is driven through the bone. Certain embodiments may include a threaded cutting region 212 immediately after the distal end 204. In certain embodiments, this threaded cutting region 212 provides the requisite interaction with bony tissue (not shown) to cut and propel the implant fixture 200 forward until the propeller 230 can interact with the bony tissue - at which time, the propeller 230 may propel the implant forward.

### Thread Form Surface Features:

FIG. 3E is a partial side perspective view of a propeller 230 of the implant fixture 200 illustrating certain distal facing or front side surface details. In contrast, FIG. 3F is a partial section view showing a cut through the threads of the propeller 230. FIG. 3G is a detailed sectional view of the thread forms where the longitudinal direction has been expanded for illustrative purposes to show additional details.

As illustrated in FIGS. 3E through 3G, in certain embodiments, there may be “pawls” or radial blades 260 projecting from the front or distal surfaces 262 of the threads 256 of the propeller 230 to create a turbine-like effect. As illustrated, in certain embodiments, the pawls 260 extend outwardly from the direction of the shaft in a somewhat radial manner, generally transverse to the circumferential direction of the thread of the propeller 230. In section, as can be seen in FIGS. 3F and 3G, the pawls 260 are generally triangular in cross-sectional shape, having a leading side or surface 266 (relative to the direction of travel 271) and a back side or surface 264 meeting to form a sharp edge 270. In certain embodiments, the sharp edge 270 may be at a predetermined angle (say 20 degrees) relative to the distal surface 262 of the thread 256 and projecting in an opposite direction from the forward rotation direction 271 (during placement of the implant fixture).

The geometry of the pawls 260 enables rotation in a single rotational direction (forward rotational travel 271) and prevents backout, similar to a ratchet and pawl mechanism, because the sharp edges 270 will catch on the surrounding bony structure if the direction of travel is reversed. In certain embodiments, there may be subtle pitch changes of the pawls 260 along the threads, which will result in increased compression of the bony material.

Focusing now on FIG. 3G, there is a detailed section view cut through one of the threads 256. In certain embodiments, a backside surface 264 of the pawl 60 is curved. The curved back side surface 264 allows additional bone harvesting because of a cupping effect, which may also create subtle compression in the harvested bony material while the thread 256 of the propeller 230 is rotating to position the implant fixture 200. With the curved back surface 264, as the thread 256 is rotated, additional pressure is applied against the bony material, creating a turbine-like effect. In other words, as the implant fixture 200 is rotating into position, the pawls 260 may create constant pressure even at low rotation speeds. The pressure may increase slightly as the implant fixture 200 progresses so that the implant fixture 200 creates compression and retains itself as it is placed. Furthermore, as rotation occurs, subtle pitch changes will occur along the threads, resulting in an increase in compression, and the “scooped” segments created by the backside of the pawls maximize surface area for bone ingrowth after placement.

In certain embodiments, the pawls may be evenly spaced throughout the faces of the threads. In other embodiments, the pawls may be spaced differently on the distal and proximal thread portions. For instance, the pawl spacing may increase along the thread flight from the distal threads to the proximal threads. In another embodiment, the pawl spacing may decrease along the thread flight from the distal to proximal threads. In addition, the pawl depth (from the face of the thread) and radial angle may be adjusted as necessary for the specific application. In yet other embodiments, the cross-section shape of the pawls may be a right triangle, an isosceles triangle, or another polygon. Furthermore, in some embodiments, the pawl projection path in the radial direction may be straight, as illustrated, or curved.

FIG. 3H illustrates an alternative embodiment for the pawl and turbine system discussed above and shows the perimeter edge 276′ of a single thread 256′ of an alternative propeller 230'. As illustrated in FIG. 3H, the pawls or radial blades 260′ may be more defined than in the previous embodiment. Similar to pawls 260 discussed above, the pawls 260′ project from the front or distal surfaces 262′ of the thread 256′ to create a turbine-like effect. As illustrated, in certain embodiments, the pawls 260′ extend from the surface 262′ of the thread 256′ towards the distal end of the shaft 202 and generally transverse to the circumferential direction of the thread of the propeller 230. In section, as can be seen in FIG. 3H, the pawls 260′ form a sharp edge 270′ which faces away from the rotational direction of travel indicated by arrow 271. In certain embodiments, the sharp edge 270′ may be at a predetermined angle (say 20 degrees) relative to the distal surface 262′ of the thread 256′.

The geometry of the pawls 260′ enables rotation in a single rotational direction 271 (forward rotational travel) and prevents backout, similar to a ratchet and pawl mechanism, because the sharp edges 270′ will catch on the surrounding bony structure if the direction is reversed. In certain embodiments, there may be subtle pitch changes of the pawls 260′ along the threads, which will result in increased compression of the bony material.

In certain embodiments, a backside surface 264′ of the pawl 260′ is curved or straight. The curved back side surface 264′ allows additional bone harvesting because of a cupping effect, which may also create subtle compression in the harvested bony material while the thread 256′ of the propeller 230 is rotating to position the implant fixture 200. With the curved back surface 264', as the thread 256′ is rotated, additional pressure is applied against the bony material, creating a turbine-like effect.

FIG. 3I is a partial side perspective view of a propeller 230 of the implant fixture 200, illustrating certain proximal facing or backside surface details of the threads 256. Turning now to FIG. 3I and FIG. 3G, the back side or proximal face 272 of the thread 256 is illustrated with a circumferential rim 274 projecting in a proximal direction around the perimeter 276 of the thread 256 as illustrated most prominently in FIG. 3G. During rotation occurring during implant placement, the rim 274 may gather additional bony material between the threads causing a pressure wave within the bony material resulting in additional compression of the bony material. In other embodiments, the proximal surface of the threads may be curved, which helps in creating a backside wave geometry that wedges the implant into the bone and increases compression.

### Flowable Material Distribution System:

FIG. 3D is an isometric section view of the implant fixture 200. In contrast, FIG. 3J is an isometric section view of the implant fixture 200, where the interior solid material is shown as transparent to illustrate certain interior passageways and details. FIG. 3K is a side view of the implant fixture 200, where the solid material is shown in a semi-transparent manner to further illustrate the interior passageways and details.

Certain embodiments may include an internal labyrinth structure having a branched, tree-like appearance for distributing biologics and bone growth after implant placement. In certain embodiments, the structure 280 is formed from a plurality of generally longitudinal tubes or cannulas 282 having initial or feed apertures 284 defined around the circular receiving aperture 222 of the central bore 220. A plurality of tubules or channels 286 branch off the longitudinal cannulas 282 and end in apertures 288 defined within the lattice 210 (see, e.g., FIGS. 3E and 3F) and or, in some embodiments, the threads 208 surrounding the shaft 202.

In certain embodiments, the initial diameter or size of the cannulas 282 varies along the longitudinal axis to allow for relatively even distribution of the flowable biologic material. For instance, distal cannula portions and distal tubules may have larger diameters where the pressure in one of the primary cannulas is less to allow the biologics to flow evenly throughout the entire structure. In certain embodiments, the cannulas and tubules may be helical and surround the center axis 214 and central bore 220, then branch or split into smaller tubules and exit at apertures defined in the minor diameter 232 of the threaded shaft 202 as illustrated in FIGS. 3D and 3E. In yet other embodiments, the tubules 286 extend into the thread forms (not shown). The tubule size and distribution may be customized for optimal graft flow - depending on the application and the flowability of the injected material.

In certain embodiments, pressurized biological material, such as treated cadaverous bone material, may be injected into the central bore 220 from a syringe or another pressure inducing device. The material will then flow down the center bore 220 into the primary cannulas 282 via the apertures 284 defined in the walls of the center bore 220 and then into the various branches or tubules 286 of the labyrinth. The pressurized material continues to flow until the material flows out of the apertures 288 defined in the minor diameter 232 of the shaft 202.

In certain embodiments, the dentist may inject certain medicines, such as analgesics or antibiotics, into the cannula to relieve post-operative discomfort and/or to prevent infection.

### Manufacturing Methods and Materials:

In certain embodiments, the implants (such as implant fixture 200) may be manufactured utilizing three-dimensional (3D) printing, where the implant is printed as a relatively complete assembly incorporating the external thread form and internal implant fixtures 200. If necessary, such embodiments may then be finalized with standard machining methods to clean up or add various surfaces and features.

In certain embodiments, the implants and implant fixtures discussed above may be fabricated from any number of biocompatible implantable materials, including but not limited to Titanium Alloys (Ti 6Al4V ELI, for example), commercially pure titanium, Chromium Cobalt (Cr—Co), stainless steels, and ceramics. In other embodiments, the implants and implant fixtures may also be manufactured from polymer, including Carbon Fiber Reinforced Polymer (“CFRP”) with a high carbon mass percentage. In some embodiments, the implants (or portions of the implants) may be coated with a bone-conducting surface treatment to increase the potential of bone on-, through-, or in-growth.

### Operation

Referring now to the above figures, the manner of using one embodiment of the present invention will now be described. After administering local anesthesia to numb the area, the dentist makes a small incision in the gum tissue to expose the underlying bone at the site of the planned implant fixture 200. Using a series of increasingly larger pilot drills, the surgeon carefully osteotomizes (drills into) the bony area, creating an appropriately sized hole for the dimensions of the implant fixture 200. Once the osteotomy is prepared, the implant fixture 200 is inserted into the prepared bone site using a special implant driver or wrench, which has a torque feature designed to mate with and apply torque on the torque engagement section 224 of the central bore 220 of the feature of the implant fixture 200. Applying controlled torque with the driver rotates a propeller of the implant fixture 200. As the implant fixture 200 is rotated, a plurality of pawls projecting from the distal surface of helical threads act as a turbine and propel the implant fixture 200 forward while causing compression on the surrounding bone. As described above, a rotation of a rim and curved surface on the proximal side of the threads tends to harvest some of the surrounding bony tissue between the threads, causing compression between the implant and the bony structure surrounding the implant.

In certain procedures, the surgeon may verify the depth and stability of the implant during placement, sometimes using intraoperative imaging. After the implant is seated, the driver is removed, and, in certain embodiments, analgesics, antibiotics, and/or bone growth material is injected into a labyrinth defined within the implant fixture 200.

The gum tissue is sutured back over the site, often with a temporary cover or healing cap placed on the implant to protect it during healing. During the osseointegration period, typically spanning several months, the bone gradually fuses to the implant surface and labyrinth, providing a solid anchor for the subsequent attachment of an abutment via an attachment mechanism such as the screw described above. After osseointegration has occurred and the abutment has been seated, a prosthetic may now be attached to the abutment.

### Advantages of Certain Embodiments

Conventional dental implants have predominantly focused on optimizing mechanical fixation through design modifications; however, biological factors such as bone quality and the interface's healing potential significantly influence their stability. Insufficient osseointegration can lead to loosening, micro-movement, and eventual failure of the implant, particularly in cases involving osteoporotic or compromised bone. The disclosed propulsor is designed to penetrate both osteopenia bone and sclerotic bone without the need for the use of percussive force. Furthermore, lattice

The compressive features of the thread form in the propeller region discussed above may cause subtle compression during the application of a smooth force used to insert the propeller. In certain situations, subtle compression may be preferred because the bony structure may not be able to withstand aggressive compression. Not only does the insertion of the propeller initially cause compression of the bony material immediately surrounding the propeller, compression continues after placement - resulting in more rapid healing when compared to conventional dental implants.

In sum, by applying controlled compression, the disclosed system can increase circumferential bone contact, promote dense bone-screw apposition, and stimulate early and robust osseointegration. This biomechanical strategy not only stabilizes the implant immediately post-surgery but also fosters favorable biological responses conducive to long-term integration.

It should be emphasized that while the present invention is primarily described in the context of a dental implant system, its innovative features and components have far-reaching potential beyond dental applications. The unique screw threads, pawls, and compression systems disclosed in this invention possess versatile characteristics that make them suitable for a wide array of medical and surgical applications throughout the human body. These components can be adapted and utilized in various orthopedic procedures, such as bone fracture fixation, spinal fusion, and joint replacement surgeries. The novel design elements of these components offer enhanced stability, improved load distribution, and optimized tissue integration, which are beneficial in numerous anatomical locations and diverse clinical scenarios.

The foregoing description of the embodiments of the invention has been presented for the purposes of illustration and description. It is not intended to be exhaustive or to limit the invention to the precise form disclosed. Many combinations, modifications, and variations are possible in light of the above teaching. For instance, in certain embodiments, each of the above-described components and features may be individually or sequentially combined with other components or features and still be within the scope of the present invention. Undescribed embodiments that have interchanged components are still within the scope of the present invention. It is intended that the scope of the invention be limited not by this detailed description but rather by the claims.

For instance, in some embodiments, there is a sophisticated dental implant device designed to enhance stability, ease of placement, and integration with surrounding bone tissue, significantly advancing traditional implant methodologies.

Embodiments of the dental implant device comprise several unique components, including an advanced implant fixture. Embodiments of the implant fixture are engineered with a shaft that features a rotational axis equipped with helical threads that include a series of interspersed root valleys having an irregular and porous lattice structure, designed to promote osseointegration by encouraging bone in-growth and providing additional fixation support. Furthermore, in certain embodiments, the porosity and surface topography of the lattice structure can be designed for differing conditions, leading to enhanced osseointegration.

The threads have turbine-like pawls projecting from their distal surfaces. These pawls are angled to enable effective forward rotational movement while resisting unintentional backward rotation. This is designed so that once the implant is positioned, it remains secure, reducing the risk of displacement. The pawls may feature various configurations, including angled, concave, or sharp edges, tailored to compress the bone and enhance grip during the implantation process, and thereafter.

Some embodiments include a channel system for bone-growth material delivery. These channels are longitudinally defined within the implant fixture, with design variations allowing for intake apertures and multiple output points along the shaft. This system enables the uniform delivery of bone growth materials directly into the surrounding bone structure, promoting enhanced integration and stability over time.

During placement, torque is applied to rotate the propeller within the bone. This motion, aided by the rear-facing pawls, compresses the bone material, enhancing initial stability. Furthermore, the device's design allows for the injection of bone growth material, channeled through an internal labyrinth to reach specific areas, maximizing biological integration and facilitating faster recovery.

In conclusion, this invention may provide a comprehensive, in-situ solution for dental implants, utilizing advanced mechanical and material distribution designs to improve implant success rates, accelerate the healing process, and ensure a lasting bond with the bony structure.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

What is claimed is:

1. A dental implant device, comprising:
an abutment; and
an implant fixture, including:
a shaft having a rotational axis;
a propeller like region including:
helical threads positioned about a longitudinal portion of the shaft forming a plurality of helical root valleys between crests of the helical threads, and the helical threads having distal surfaces and proximal surfaces;
a plurality of pawls projecting from the distal surfaces to create a turbine-like thread-form shape;
wherein the plurality of pawls are angled with respect to the distal surfaces to allow for forward rotation of the threads and to resist backward rotation of the threads;
an irregular lattice structure formed within the plurality of helical root valleys; and
a means for attaching the abutment to the implant fixture.

2. The dental implant device of claim 1, wherein at least one pawl in the plurality of pawls has an angled surface facing a forward rotation direction and a concave curved surface facing away from the forward rotation direction.

3. The dental implant device of claim 1, wherein at least one pawl in the pluralities of pawls has a sharp edge projecting away from a direction of forward rotation and towards a direction of backward rotation.

4. The dental implant device of claim 1, wherein at least one pawl in the plurality of pawls has an angled surface facing a forward rotation direction and a concave curved surface facing away from the forward rotation direction.

5. The dental implant device of claim 1, wherein at least one pawl in the plurality of pawls has an angled surface facing a forward rotation direction and a straight surface facing away from the forward rotation direction.

6. The dental implant device of claim 1, wherein at least one pawl in the plurality of pawls has an angled surface facing a forward rotation direction and an angled surface facing away from the forward rotation direction.

7. The dental implant device of claim 1, wherein at least one thread of the helical threads includes a circumferential rim projecting from the proximal surface of the threads.

8. The dental implant device of claim 1, wherein the proximal surfaces of the helical threads have a cross-sectional shape that is curved in a concave manner.

9. The dental implant device of claim 1, further including at least one longitudinal channel defined within the implant fixture having an intake aperture defined in a proximal portion of the implant fixture and at least one output aperture defined along the shaft positioned distally from the intake aperture.

10. The dental implant device of claim 1, further including at least one longitudinal channel defined within the implant fixture having an intake aperture defined in a proximal portion of the implant fixture and a plurality of tubules branching from the at least one longitudinal channel and ending in a plurality of output apertures defined along the shaft and positioned distal to the intake aperture.

11. The dental implant device of claim 10, wherein a cross-sectional area of the longitudinal channel varies along its longitudinal length.

12. The dental implant device of claim 10, wherein cross-section areas of the tubules are variable in size to allow for a uniform deposition of material through the plurality of output apertures.

13. The dental implant device of claim 10, wherein the intake aperture is defined within a central bore located defined within the proximal end.

14. The dental implant device of claim 1, wherein the implant fixture comprises a distal portion and a proximal portion, wherein the distal portion includes a forward distal thread-form shape to assist in drilling through bony tissue during implant placement.

15. An implant fixture for a dental implant device, comprising:
a propeller like region comprising:
a shaft having a rotational axis;
helical threads positioned about a longitudinal portion of the shaft forming a plurality of helical root valleys between crests of the helical threads, and the helical threads having distal surfaces and proximal surfaces;
a plurality of pawls projecting from the distal surfaces to create a turbine-like thread-form shape;
wherein the plurality of pawls are angled with respect to the distal surfaces to allow for forward rotation of the threads and to resist backward rotation of the threads;
the helical threads include a circumferential rim projecting from the proximal surface of the threads;
the proximal surfaces of the helical threads have a cross-sectional shape that is curved in a concave manner; and
an irregular lattice structure formed within the plurality of helical root valleys.

16. A method of placing an implant fixture in bony material, the method comprising:
applying a torque to a proximal end of the implant fixture to rotate a propeller about the implant fixture's longitudinal axis in a rotational direction within a bony structure to propel the implant in a longitudinal direction and resulting in a rotation of a plurality of pawls to compress the bony material while propelling the propeller forward;
compressing bony material between a cupped proximal side of a first thread of the propeller and a distal side of a second thread of the propeller; and
injecting a internal labyrinth defined in the propeller with bone-growth material such that the bone-growth material flows from an opening defined in a proximal portion of the implant fixture into a plurality of channels and out of a plurality of openings defined along a shaft of the implant fixture.

17. The method of claim 16, wherein the injecting a internal labyrinth is injecting the bone-growth material into at least two internal longitudinal cannulas such that the bone-growth material flows from openings defined in a proximal portion of the implant fixture into a plurality of channels and out of a plurality of openings defined along a shaft of the implant fixture.
